(19) United States
(12) Patent Application Publication
(10) Pub. No.: US 2024/0216468 A1
(43) Pub. Date: Jul. 4, 2024

(54) NOVEL ANTI-INFLAMMATORY THERAPEUTICS AND METHOD OF USE THEREOF

(71) Applicant: The Regents of the University of California, Oakland, CA (US)

(72) Inventors: Elisabetta MANTUANO, La Jolla, CA (US); Steven L GONIAS, La Jolla, CA (US)

(73) Assignee: The Regents of the University of California, Oakland, CA (US)

(21) Appl. No.: 18/554,705

(22) PCT Filed: Apr. 18, 2022
(86) PCT No.: PCT/US2022/025202

§ 371 Date: Oct. 10, 2023

Related U.S. Application Data
(60) Provisional application No. 63/184,980, filed on May 6, 2021.

(51) Int. Cl. A61K 38/17; A61K 47/69; A61P 37/06

(52) U.S. Cl. CPC A61K 38/177; A61K 47/6901; A61P 37/06

(57) ABSTRACT

The present disclosure provides novel anti-inflammatory therapeutics, and method of use thereof. The disclosed anti-inflammatory therapeutics comprise soluble, membrane-anchored, and/or extracellular vesicle (EV)-associated PrP^{C}, and/or analogues thereof, that interacts with NMDA-R/LRP1 receptor complext to regulate innate immunity and provide anti-inflammatory activity in diseases in which innate immunity plays an important role.

### CROSS-REFERENCE

This application claims the benefit of U.S. Provisional Application No. 63/184,980 filed on May 6, 2021, the entire content of which is incorporated herein by reference.

### STATEMENT REGARDING FEDERALLY SPONSORED RESEARCH OR DEVELOPMENT

This invention was made with government support under HL136395 awarded by the National Institutes of Health. The government has certain rights in the invention.

### FIELD AND BACKGROUND

The present disclosure relates generally to anti-inflammatory therapies and/or regulation of innate immunity. More specifically, the present disclosure relates to cellular prion protein (PrP^{C}) and extracellular vesicle (EV)-based therapies with anti-inflammatory activity.

Cellular prion protein (PrP^{C}) is a glycosylphosphatidylinositol (GPI)-anchored membrane protein that localizes mainly in lipid rafts (1). Misfolding of PrP^{C} into the β-sheet-rich, scrapie conformation (PrP^{Sc}) causes protein aggregation and prion diseases, associated with rapid neurodegeneration (2). The physiological role of non-pathogenic PrP^{C} remains incompletely understood. PrP^{C} is expressed by diverse cell types, inside and outside the nervous system, including T-lymphocytes, natural killer cells, mast cells, and macrophages (3-6). Understanding the function of non-pathogenic PrP^{C} is an important goal.

Non-pathogenic PrP^{C} exists in at least three different states that may be relevant to its function in cell physiology. In addition to GPI-anchored PrP^{C} in cells, derivatives of PrP^{C} may be released from cells into solution by proteases in the ADAM family (7-9). PrP^{C} also may be released from cells after packaging into extracellular vesicles (EVs). EVs are produced by nearly all cells and participate in cell-cell communication (10-12). Both PrP^{C} and its misfolded isoform, PrP^{Sc}, have been identified in EVs in various body compartments, including blood (13, 14). PrP^{C} may be enriched in exosomes (15-18), a specific type of EV formed in multivesicular bodies in the endosomal transport pathway (10-12, 19).

There is substantial evidence that PrP^{C} regulates inflammation (3, 20-22). Mice treated with PrP^{C}-specific antibody are protected from the lethal effects of Influenza A virus by a mechanism that apparently involves activation of Src family kinases (SFKs) in macrophages and induction of an M2-like anti-inflammatory state in these cells (23). When the gene encoding PrP^{C} (Prnp) is deleted in mice, susceptibility to the toxic effects of lipopolysaccharide (LPS) is increased (24) and colitis is more severe following treatment with Dextran Sulfate Sodium (DSS) (25).

It is demonstrated that a soluble derivative of PrP^{C} (S-PrP), which corresponds closely in sequence to the form of PrP^{C} released by ADAM10 (7), activates cell-signaling and elicits biological responses in PC12 cells and Schwann cells by a mechanism that requires LDL Receptor-related Protein-1 (LRP1) and the N-methyl-D-aspartate receptor (NMDA-R) (26). LRP1 functions as a receptor for over 100 ligands; however, only a subset of these ligands simultaneously engage the NMDA-R to activate cell-signaling (27-29). Tissue-type plasminogen activator (tPA) and α₂-macroglobulin (α₂M) are examples of LRP1 ligands that activate cell-signaling via the NMDA-R/LRP1 receptor complex in PC12 cells and Schwann cells, similarly to S-PrP (26, 28, 29). In macrophages, binding of tPA to the NMDA-R/LRP1 receptor complex blocks the pro-inflammatory activity of agonists that activate Toll-like Receptors (TLRs), including TLR2, TLR4, and TLR9 (30-33). Thus, the NMDA-R/LRP1 receptor complex emerged as an intriguing candidate to explain the anti-inflammatory activity of PrP^{C}.

Prior to the study identifying the NMDA-R/LRP1 receptor complex as a candidate cell-signaling receptor for S-PrP (26), there was already evidence that PrP^{C} interacts with LRP1. Membrane-anchored PrP^{C} physically associates with LRP1 within the plasma membranes of neuron-like cells (34, 35). This interaction apparently controls PrP^{C} trafficking and may also regulate the ability of LRP1 to initiate cell-signaling in response to tPA. PrP^{C} has been shown to bind tPA, directly and with high affinity (36). This interaction may be important in the pathway by which membrane-anchored PrP^{C} supports tPA-initiated cell-signaling via the NMDA-R/LRP1 receptor complex (35).

### SUMMARY

The present disclosure provides a novel pathway that regulates innate immunity and involves the use of the non-pathogenic cellular prion protein (PrP^{c}) through the NMDA-R/LRP1 complex. More specifically, the present disclosure provides for the first time that membrane-anchored, soluble, and extracellular vesicle (EV)-associated PrP^{c} have uniquely broad anti-inflammatory activity and/or attenuate innate immunity by an NMDA-R-dependent mechanism, wherein NMDA-R/LPR1 receptor complex is essential for anti-inflammatory cell signaling and regulation of cytokine expression by S-PrP and EV-associated PrP^{c}. The present disclosure provides a foundation and suggestion for novel anti-inflammatory therapies by developing unique novel anti-inflammatory chemistries and/or drugs and novel EVs therapies with anti-inflammatory activity.

The present disclosure, therefore, provides a novel anti-inflammatory therapeutics, and method of use thereof. The disclosed anti-inflammatory therapeutics comprise soluble, membrane-anchored, and extracellular vesicle (EV)-associated PrP^{C}, and/or analogues thereof, that interacts with NMDA-R/LRP1 receptor complex to regulate innate immunity and provide anti-inflammatory activity in diseases in which innate immunity plays an important role, such diseases include but are not limited to, Inflammatory Bowel Disease, Rheumatoid Arthritis, Psoriasis, Chronic Pain Disorders, Neurodegenerative Disease, and Multiple Sclerosis.

In certain embodiments, the present disclosure provides that PrP^{C}, which is released from cells, opposes the activity of Pattern Recognition Receptors (PRRs). In certain embodiments, the present disclosure provides that a soluble derivative of PrP^{C} (S-PrP) counteracts inflammatory responses triggered by numerous Pattern Recognition Receptors (PRRs) in macrophages, including Toll-like Receptor (TLR)2, TLR4, TLR7, TLR9 and Nucleotide Binding Oligomerization Domain Containing-1 (NOD1) and NOD2 (30-33). S-PrP also significantly attenuates the toxicity of lipopolysaccharide (LPS) in mice. The response of macrophages to S-PrP is mediated by a receptor assembly that includes the NMDA Receptor (NMDA-R) and LRP1.

In certain embodiments, the present disclosure provides that PrP^{C} was identified in EVs isolated from human plasma. These EVs replicated the activity of S-PrP, inhibiting cytokine expression and IκBα phosphorylation in LPS-treated macrophages. The effects of plasma EVs on LPS-treated macrophages were blocked by PrP^{C}-specific antibody, by antagonists of LRP1 and the NMDA-R, by deleting Lrp1 in macrophages, and by inhibiting Src family kinases (SFKs), upstream of IκBα and ERK1/2. Treating EVs with phosphatidylinositol-specific phospholipase C (PI-PLC), an enzyme that releases GPI-anchored proteins from plasma membranes (37, 38), dissociated the LPS-regulatory activity from the EVs, rendering the EVs inactive as LPS inhibitors. The LPS-regulatory activity lost from PI-PLC-treated EVs was recovered in the solution phase, demonstrating that PrP^{C} is GPI-anchored in EVs, as it is in cells. S-PrP and EV-associated PrP^{C} regulate innate immunity similarly, via the NMDA-R/LRP1 receptor system. The scope of PRRs antagonized by S-PrP suggests that PrP^{C}, which is released from cells, may have broad anti-inflammatory activity.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Many aspects of the present disclosure can be better understood with reference to the following drawings (also “Figures” or “FIGs”). The components in the drawings are not necessarily to scale, emphasis instead being placed upon clearly illustrating the principles of the present disclosure. Moreover, in the drawings, like reference numerals designate corresponding parts throughout the several views.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

FIGS. 1A-1F. S-PrP counteracts the activity of LPS in vitro and in vivo. FIG. 1A. BMDMs from C57BL/6J mice were treated for 6 h with LPS (0.1 μg/mL) in presence of increasing concentrations of S-PrP (10-120 nM) or with 40 nM S-PrP in the absence of LPS. RT-qPCR was performed to determine mRNA levels for TNFα and IL-6 (n=3). FIG. 1B. BMDMs were stimulated for 1 h with increasing concentrations of S-PrP (20-120 nM). Cell extracts were subjected to immunoblot analysis to detect phospho-ERK1/2 and total ERK1/2. FIG. 1C. BMDMs were treated for 1 h with LPS (0.1 μg/mL) in presence of increasing concentrations of S-PrP (20-120 nM). Immunoblot analysis was performed to detect phospho-IκBα, total IκBα, and β-actin. FIG. 1D. pMacs were treated with LPS (0.1 μg/mL), EI-tPA (12 nM), S-PrP (40 nM), LPS plus EI-tPA, LPS plus S-PrP, or vehicle for 6 h. RT-qPCR was performed to compare mRNA levels for TNFα and IL-6 (n=3). (E) Sepsis scores are shown for wild-type C57BL/6J mice treated by IV injection with vehicle (n=7) or 2.5 μg/g S-PrP (n=6), 1 h after IP injection of LPS at 1.5 times the LD₅₀ (9 mg/kg). FIG. 1F. Kaplan-Meier survival curves are shown for mice treated by IV injection with 2.5 μg/g S-PrP (n=6) or vehicle (n=7), 1 h after IP injection of LPS at 1.5 times the LD₅₀ (9 mg/kg). Significance was determined by Mantel-Cox test. RT-qPCR and sepsis scoring data are expressed as the mean±SEM in panels (A), (D) and (E) (one-way ANOVA; *P<0.05, **P<0.01, ***P<0.001, ****P<0.0001, n.s.: not statistically significant).

FIGS. 2A-2G. S-PrP inhibits multiple Pattern Recognition Receptors. FIG. 2A. BMDMs were treated with the TLR2 agonist LTA (1.0 μg/mL), S-PrP (40 nM), or LTA plus S-PrP for 6 h. RT-qPCR was performed to compare mRNA levels of TNFα and IL-6 (n=4). FIG. 2B. BMDMs were treated with the TLR9 agonist, ODN 1826 (1.0 μM), S-PrP (40 nM), or ODN 1826 plus S-PrP for 6 h. RT-qPCR was performed to compare mRNA levels of TNFα and IL-6 (n=3). FIG. 2C. BMDMs were treated with the TLR7 agonist IMQ (3.0 μg/mL), S-PrP (40 nM), or IMQ plus S-PrP for 6 h. RT-qPCR was performed to compare mRNA levels of TNFα and IL-6 (n=3). FIG. 2D. BMDMs were treated with the NOD1 agonist C12-iE-DAP (1 μg/mL), S-PrP (40 nM), or C12-iE-DAP plus S-PrP for 6 h. RT-qPCR was performed to compare mRNA levels of TNFα and IL-6 (n=3). FIG. 2E. BMDMs were treated with the NOD2 agonist, L18-MDP (0.1 μg/mL), S-PrP (40 nM), or L18-MDP plus S-PrP for 6 h. RT-qPCR was performed to compare mRNA levels of TNFα and IL-6 (n=3). Data are expressed as the mean±SEM (one-way ANOVA; ***P<0.001, ****P<0.0001). FIGS. 2F-2G. Immunoblot analysis showing total and phosphorylated IκBα in BMDMs treated for 1 h with: (F) C12-iE-DAP (1.0 μg/mL) in presence or absence of S-PrP (40 nM); and (G) L18-MDP (0.1 μg/mL) in presence or absence of S-PrP (40 nM). β-actin levels are shown as a control for load.

FIGS. 3A-3B. Effects of PrP^{C}-specific monoclonal antibodies on the activity of S-PrP. FIG. 3A. BMDMs were treated for 1 h with LPS (0.1 μg/mL), LPS plus S-PrP (40 nM), or vehicle in the presence of POM1, POM2, POM3, POM19, or nonspecific IgG (10 μg/ml). Immunoblot analysis was performed to detect phospho-IκBα, total IκBα, and β-actin. FIG. 3B. BMDMs were treated for 6 h with LPS (0.1 μg/mL), LPS plus S-PrP (40 nM), or with vehicle in presence or absence of POM2 antibody (10 μg/ml). RT-qPCR was performed to compare mRNA levels for TNFα and IL-6 (n=3). RT-qPCR data are expressed as the mean±SEM (one-way ANOVA; **P<0.01, ***P<0.001, ****P<0.0001, n.s.: not statistically significant).

FIGS. 4A-4F. The NMDA-R/LRP1 system mediates the anti-LPS/TLR4 activity of S-PrP. FIG. 4A. BMDMs were pre-incubated with MK801 (1.0 μM), DXM (10 μM), or vehicle for 30 min. The cells were then treated with LPS (0.1 μg/mL), S-PrP (40 nM), LPS plus S-PrP, or with vehicle for 6 h. RT-qPCR was performed to compare mRNA levels for TNFα and IL-6 (n=3). FIG. 4B. BMDMs were pre-treated with MK801 (1.0 μM), DXM (10 μM), or vehicle for 30 min, and then with LPS (0.1 μg/mL) and increasing concentrations of S-PrP (40-120 nM) for 1 h, as indicated. Immunoblot analysis was performed to detect phospho-IκBα, total IκBα, and β-actin. FIG. 4C. BMDMs were pre-treated with RAP (150 nM) or vehicle for 30 min, and then with LPS (0.1 μg/mL) and S-PrP (40 nM) for 6 h, as indicated. RT-qPCR was performed to compare mRNA levels for TNFα and IL-6 (n=3). FIG. 4D. BMDMs from mLrp1^{+/+} and mLrp1^{-/-} mice were treated for 6 h with LPS (0.1 μg/mL) in presence of increasing concentrations of S-PrP (40-120 nM). RT-qPCR was performed to determine mRNA levels for TNFα (n=3). FIG. 4E. BMDMs from mLrp1^{+/+} and mLrp1^{-/-} mice were treated for 6 h with LPS (0.1 μg/mL) in presence of increasing concentrations of S-PrP (40-120 nM). RT-qPCR was performed to determine mRNA levels for IL-6 (n=3). FIG. 4F. BMDMs from mLrp1^{-/-} mice were treated for 1 h with LPS (0.1 μg/mL) in the presence of increasing concentrations of S-PrP (20-120 nM). Immunoblot analysis was performed to detect phospho-IκBα, total IκBα, and β-actin. RT-qPCR data are expressed as the mean±SEM (one-way ANOVA; *P<0.05, ****P<0.0001, n.s.: not statistically significant).

FIGS. 5A-5B. Macrophage PrP^{C} is not necessary for the anti-LPS/TLR4 activity of S-PrP. BMDMs isolated from WT mice (FIG. 5A) and from Prnp^{-/-} mice (FIG. 5B) were treated with LPS (0.1 μg/mL), S-PrP (40 nM), LPS plus S-PrP, or vehicle for 6 h. RT-qPCR was performed to compare mRNA levels for TNFα and IL-6 (n=3). Data are expressed as the mean±SEM (one-way ANOVA; ****P<0.0001).

FIGS. 6A-6D. Human plasma EVs inhibit the response of macrophages to LPS. FIG. 6A. NTA of a representative human plasma EV preparation obtained by sequential UC. FIG. 6B. Representative TEM images of EVs isolated from human plasma (Scale bar=50 nm). FIG. 6C. Characterization of two representative human plasma EV UC preparations. Immunoblot analysis was performed to detect the exosome biomarkers, flotillin and Tsg101. The same blots were probed for PrP^{C} and GM130.FIG. 6D. BMDMs were treated for 1 h with LPS (0.1 μg/mL) in presence of increasing concentrations of human plasma EVs (0.1-2.0 μg/mL). Immunoblot analysis was performed to detect phospho-IκBα, total IκBα, and β-actin.

FIGS. 7A-7D. The NMDA-R/LRP1 receptor complex is required for the anti-LPS/TLR4 activity of human plasma EVs. FIG. 7A. BMDMs were pre-incubated with MK801 (1.0 M), RAP (150 nM), or vehicle for 30 min. The cells were then treated with LPS (0.1 μg/mL), human plasma EVs (1.0 μg/mL), LPS plus EVs, or with vehicle for 6 h. RT-qPCR was performed to compare mRNA levels for TNFα and IL-6 (n=3). FIG. 7B. BMDMs were pre-treated with MK801 (1.0 μM), RAP (150 nM), or vehicle for 30 min, and then with LPS (0.1 μg/mL) and/or human plasma EVs (1.0 μg/mL) for 1 h, as indicated. Immunoblot analysis was performed to detect phospho-IκBα, total IκBα, and β-actin. FIG. 7C. BMDMs from mLrp1^{-/-} mice were treated for 6 h with LPS (0.1 μg/mL), human plasma EVs (1.0 μg/mL), or LPS plus EVs. RT-qPCR was performed to determine mRNA levels for TNFα and IL-6 (n=3). FIG. 7D. BMDMs from mLrp1^{-/-} mice were treated with LPS (0.1 μg/mL), human EVs (1.0 μg/mL), LPS plus EVs, or with vehicle for 1 h. Immunoblot analysis was performed to detect phospho-IκBα, total IκBα, and β-actin. RT-qPCR data are expressed as the mean±SEM (one-way ANOVA; *P<0.05, **P<0.01, ***P<0.001, ****P<0.0001, n.s.: not statistically significant).

FIGS. 8A-8F. GPI-anchored PrP^{C} is responsible for the anti-LPS/TLR4 activity of human plasma EVs. FIG. 8A. BMDMs were treated for 6 h with LPS (0.1 μg/mL), human EVs (1.0 μg/mL), LPS plus EVs, or vehicle in the presence of POM1, POM2, POM3, or POM19 (10 μg/ml). RT-qPCR was performed to determine TNFα mRNA expression (n=3). FIG. 8B. BMDMs were treated for 1 h with LPS (0.1 μg/mL), human plasma EVs (1.0 μg/mL), LPS plus EVs, or vehicle in the presence of nonspecific IgG, POM1, POM2, POM3, or POM19 (10 μg/ml). Immunoblot analysis was performed to detect phospho-IκBα, total IκBα, and β-actin. FIG. 8C. BMDMs were treated with LPS (0.1 μg/mL) in the presence or absence of α₂M (10 nM), EI-tPA (12 nM), and POM2 (10 μg/mL) for 6 h, as indicated. RT-qPCR was performed to determine TNFα and IL-6 mRNA expression (n=3). FIG. 8D. BMDMs were treated with LPS (0.1 μg/mL), α₂M (10 nM), and POM2 (10 μg/mL) for 1 h, as indicated. Immunoblot analysis was performed to detect phospho-IκBα, total IκBα, and β-actin. FIG. 8E. Human plasma EVs were treated with PI-PLC or with vehicle and subjected to UC to separate the EVs from soluble proteins in the supernatants. BMDMs were treated for 1 h with LPS (0.1 μg/mL), 2.0 μg/mL of EVs that were unmodified (Control), LPS+unmodified EVs, 2.0 μg/mL of PI-PLC-treated EVs (PI-PLC), or LPS+PI-PLC-treated EVs, as indicated. Immunoblot analysis was performed to detect phospho-IκBα, total IκBα, and β-actin. FIG. 8F. Human plasma EVs were treated with PI-PLC or with vehicle. The supernatants were separated from the EVs by UC and studied. BMDMs were treated for 1 h with LPS (0.1 μg/mL), supernatants harvested from 2.0 μg of control EVs (Control), LPS +supernatants from control EVs, supernatants from 2.0 μg of PI-PLC-treated EVs (PI-PLC), or LPS+supernatants from PI-PLC-treated EVs, as indicated. Immunoblot analysis was performed to detect phospho-IκBα, total IκBα, and β-actin. RT-qPCR data are expressed as the mean±SEM (one-way ANOVA; ****P<0.0001, n.s.: not statistically significant).

FIGS. 9A-9D. Inhibiting SFKs blocks the anti-LPS-TLR4 activity of human plasma EVs. FIG. 9A. BMDMs from wild-type mice were treated with human plasma EVs (4 μg/mL), for the indicated times. Phospho-SFKs and total SFKs were determined by immunoblot analysis. FIG. 9B. BMDMs were treated with human plasma EVs (4 μg/mL), MK801 (1.0 μM), PP2 (1.0 μM), EVs plus MK801 or EVs plus PP2 for 1 h, as indicated. Phospho-ERK1/2 and total ERK1/2 were determined by immunoblot analysis. FIG. 9C. BMDMs were treated with LPS (0.1 μg/mL), EVs (1.0 μg/mL), and/or PP2 (1.0 μM) for 1 h, as indicated. Immunoblot analysis was performed to detect phospho-IκBα and total IκBα. FIG. 9D. BMDMs were treated for 6 h with LPS, EVs, and/or PP2, as indicated. RT-qPCR was performed to determine mRNA levels for TNFα and IL-6. RT-qPCR data are expressed as the mean±SEM (n=3; one-way ANOVA; ***P<0.01, ****P<0.0001).

FIGS. 10A-10C. S-PrP does not affect BMDM viability. BMDMs were treated with S-PrP (FIG. 10B: 40 nM and FIG. 10C: 120 nM) or vehicle (FIG. 10A) for 6 h. Representative flow cytometry analyses to detect apoptotic cells are shown. Apoptotic BMDMs were detected as cells that capture high levels of cell-surface Annexin V (y-axis, top of graph). 7-AAD uptake is shown on the x-axis. 7-AAD exclusion is a property of viable cells and may also be used to distinguish early apoptotic cells from late apoptotic or dead cells.

FIG. 11. MK801 and DXM do not affect the ability of LPS to induce IκBαphosphorylation in the absence of S-PrP. BMDMs were pre-treated with MK801 (1.0 μM), DXM (10 μM), or vehicle for 30 min, and then with LPS (0.1 μg/mL) for 1 h, as indicated. Immunoblot analysis was performed to detect phospho-IκBα, total IκBα, and β-actin.

### DETAILED DESCRIPTION

The present disclosure provides a novel anti-inflammatory therapeutics, and method of use thereof. The disclosed anti-inflammatory therapeutics comprise soluble, membrane-anchored, and extracellular vesicle (EV)-associated PrP^{C}, and/or analogues thereof, that interacts with NMDA-R/LRP1 receptor to regulate innate immunity and provide anti-inflammatory activity in diseases in which innate immunity plays an important role. Such diseases include, but are not limited to, including, but not limited to, inflammatory Bowel Disease, Rheumatoid Arthritis, Psoriasis, Chronic Pain Disorders, Neurodegenerative Disease, and Multiple Sclerosis.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Aspects of the present disclosure will employ, unless otherwise indicated, techniques of molecular biology, microbiology, organic chemistry, biochemistry, physiology, cell biology, blood vessel biology, and the like, which are within the skill of the art. Such techniques are explained fully in the literature.

Prior to describing the various aspects of the present disclosure, the following definitions are provided and should be used unless otherwise indicated. Additional terms may be defined elsewhere in the present disclosure.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used herein, the term “and/or” includes any and all combinations of one or more of the associated listed items. Expressions such as “at least one of,” when preceding a list of elements, modify the entire list of elements and do not modify the individual elements of the list.

As used in the specification and the appended claims, the singular forms “a,” “an” and “the” include plural referents unless the context clearly dictates otherwise. Reference to “a/an” chemical compound, therapeutic agent, and pharmaceutical composition each refers to one or more molecules of the chemical compound, therapeutic agent, and pharmaceutical composition rather than being limited to a chemical compound, therapeutic agent, and pharmaceutical composition, the one or more molecules may or may not be identical, so long as they fall under the category of the chemical compound, therapeutic agent, and pharmaceutical composition. Thus, for example, “a” therapeutic agent is interpreted to include one or more molecules of the therapeutic agent, where the therapeutic agent molecules may or may not be identical (e.g., comprising different isotope abundances and/or different degrees of hydration or in equilibrium with different conjugate base or conjugate acid forms).

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double-and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term “treatment” as used herein can include any treatment of inflammation associated with any disease in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C1-to-C6 alkyl esters and C5-to-C7 cycloalkyl esters, although C1-to-C4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C1-to-C6 alkyl amines and secondary C1-to-C6 dialkyl amines. In the case of secondary amines, the amine can also be in the form of a 5- or 6-membered heterocycle containing one nitrogen atom. Amides derived from ammonia, C1-to-C3 alkyl primary amides and C1-to-C2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

It is understood, that unless otherwise specified, temperatures referred to herein are based on atmospheric pressure (i.e. one atmosphere).

### Novel anti-inflammatory chemistries, drugs and/or EV-associated PrP^{c}

As disclosed herein, in certain embodiments, the present disclosure provides novel therapeutics comprising a soluble, membrane-anchored, and/or extracellular vesicle (EV)-associated PrP^{C}, and/or analogues thereof, that interacts with NMDA-R/LRP1 receptor complex to regulate innate immunity and provide anti-inflammatory activity in diseases in which innate immunity plays an important role. Compositions comprising the novel therapeutics that comprise a soluble, membrane-anchored, and/or extracellular vesicle (EV)-associated PrP^{C}, and/or analogues thereof, that interacts with NMDA-R/LRP1 receptor complex to regulate innate immunity and provide anti-inflammatory activity are also disclosed herein.

### Treatment and/or Prevention Method

Disclosed herein is a method of treating and/or preventing inflammation in various disease in a patient in need thereof, comprising administering to the patient a therapeutically effective amount of a soluble, membrane-anchored, and/or extracellular vesicle (EV)-associated PrP^{C}, and/or analogues thereof, that interacts with NMDA-R/LRP1 receptor complex to regulate innate immunity and provide anti-inflammatory activity in diseases in which innate immunity plays an important role. In certain embodiments, such diseases include but are not limited to, Inflammatory Bowel Disease, Rheumatoid Arthritis, Psoriasis, Chronic Pain Disorders, Neurodegenerative Disease, and Multiple Sclerosis.

In certain embodiments, provided herein is a method for reducing the level of inflammation. Methods for measuring the extent of inflammation are well known in the art. In one embodiment, the level of inflammation is reduced by about 5% to about 100%. In one embodiment, the level of inflammation is reduced by at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, at least about 95%, or about 100% in the subject.

### Dosing and Administration

While it is possible for an active ingredient to be administered alone, it may be preferable to present them as pharmaceutical formulations or pharmaceutical compositions as described below. The formulations, both for veterinary and for human use, of the disclosure comprise at least one of the active ingredients, together with one or more acceptable carriers therefor and optionally other therapeutic ingredients. The carriers must be “acceptable” in the sense of being compatible with the other ingredients of the formulation and physiologically innocuous to the recipient thereof.

Each of the active ingredients can be formulated with conventional carriers and excipients, which will be selected in accord with ordinary practice. Tablets can contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11 but is ordinarily about 7 to 10. The therapeutically effective amount of active ingredient can be readily determined by a skilled clinician using conventional dose escalation studies. Typically, the active ingredient will be administered in a dose from 0.01 milligrams to 2 grams. In one embodiment, the dosage will be from about 10 milligrams to 450 milligrams. In another embodiment, the dosage will be from about 25 to about 250 milligrams. In another embodiment, the dosage will be about 50 or 100 milligrams. In one embodiment, the dosage will be about 100 milligrams. It is contemplated that the active ingredient may be administered once, twice or three times a day. Also, the active ingredient may be administered once or twice a week, once every two weeks, once every three weeks, once every four weeks, once every five weeks, or once every six weeks.

The pharmaceutical composition for the active ingredient can include those suitable for the foregoing administration routes. The formulations can conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations suitable for oral administration can be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste. In certain embodiments, the active ingredient may be administered as a subcutaneous injection.

A tablet can be made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, or surface active agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

The active ingredient can be administered by any route appropriate to the condition. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. In certain embodiments, the active ingredients are orally bioavailable and can therefore be dosed orally. In one embodiment, the patient is human.

When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as, for example, calcium or sodium carbonate, lactose, lactose monohydrate, croscarmellose sodium, povidone, calcium or sodium phosphate; granulating and disintegrating agents, such as, for example, maize starch, or alginic acid; binding agents, such as, for example, cellulose, microcrystalline cellulose, starch, gelatin or acacia; and lubricating agents, such as, for example, magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as, for example, glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as, for example, peanut oil, liquid paraffin or olive oil.

Aqueous suspensions of the disclosure contain the active materials in admixture with excipients suitable for the manufacture of aqueous suspensions. Such excipients include a suspending agent, such as, for example, sodium carboxymethylcellulose, methylcellulose, hydroxypropyl methylcelluose, sodium alginate, polyvinylpyrrolidone, gum tragacanth and gum acacia, and dispersing or wetting agents such as, for example, a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as, for example, ethyl or n-propyl p-hydroxy-benzoate, one or more coloring agents, one or more flavoring agents and one or more sweetening agents, such as, for example, sucrose or saccharin.

Oil suspensions may be formulated by suspending the active ingredient in a vegetable oil, such as, for example, arachis oil, olive oil, sesame oil or coconut oil, or in a mineral oil such as, for example, liquid paraffin. The oral suspensions may contain a thickening agent, such as, for example, beeswax, hard paraffin or cetyl alcohol. Sweetening agents, such as, for example, those set forth above, and flavoring agents may be added to provide a palatable oral preparation. These compositions may be preserved by the addition of an antioxidant such as, for example, ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as, for example, olive oil or arachis oil, a mineral oil, such as, for example, liquid paraffin, or a mixture of these. Suitable emulsifying agents include naturally-occurring gums, such as, for example, gum acacia and gum tragacanth, naturally occurring phosphatides, such as, for example, soybean lecithin, esters or partial esters derived from fatty acids and hexitol anhydrides, such as, for example, sorbitan monooleate, and condensation products of these partial esters with ethylene oxide, such as, for example, polyoxyethylene sorbitan monooleate. The emulsion may also contain sweetening and flavoring agents. Syrups and elixirs may be formulated with sweetening agents, such as, for example, glycerol, sorbitol or sucrose. Such formulations may also contain a demulcent, a preservative, a flavoring or a coloring agent.

The pharmaceutical compositions of the disclosure may be in the form of a sterile injectable preparation, such as, for example, a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as, for example, a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as, for example, oleic acid may likewise be used in the preparation of injectables.

The amount of active ingredient that may be combined with the carrier material to produce a single dosage form will vary depending upon the host treated and the particular mode of administration, such as oral administration or subcutaneous injection. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur. When formulated for subcutaneous administration, the formulation is typically administered about twice a month over a period of from about two to about four months.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations can be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

In certain embodiments, the inhibitor of the present disclosure may be formulated in any suitable dosage form for an appropriate administration. In certain embodiments, the methods provided herein comprise administering a pharmaceutical composition comprising the inhibitor of the present disclosure and a pharmaceutically acceptable carrier or excipient. Combination formulations and/or treatment according to the present disclosure comprise the inhibitor of the present disclosure together with one or more pharmaceutically acceptable carriers or excipients and optionally other therapeutic agents, now known or later developed, for treating and/or preventing inflammation. Combination formulations containing the active ingredient may be in any form suitable for the intended method of administration.

The present disclosure provides a novel pathway by which PrP^{C} may express anti-inflammatory activity when it is released from cells as a soluble derivative or as an EV-associated protein. The NMDA-R and LRP1 are central components of this pathway. These receptors were previously shown to be res-ponsible for the anti-inflammatory activity of tPA in macrophages (30, 31, 33). The NMDA-R and LRP1 are considered as candidates for mediating the response to S-PrP in macrophages because of their known role in S-PrP-initiated cell-signaling in PC12 cells and Schwann cells (26). The activity of the NMDA-R and LRP1, as receptors for EV-associated PrP^{C}, was unanticipated because EV-associated proteins are not previously described as ligands for LRP1 or the NMDA-R. Inflammatory cells in addition to macrophages express the NMDA-R and LRP1 (55). Thus, the NMDA-R/LRP1 receptor system may mediate the anti-inflammatory activity of shed and EV-associated PrP^{C} derivatives, in vivo, in target cells in addition to macrophages.

S-PrP blocked inflammatory cytokine mRNA expression in macrophages in response to ligands that activate diverse TLRs and also inhibited TLR-induced IκBα phosphorylation. S-PrP inhibited the toxicity of LPS in vivo. Furthermore, S-PrP directly activated ERK1/2 in cultured macrophages, in the absence of TLR agonists. These S-PrP activities were similar to those demonstrated previously for EI-tPA, which also engages the NMDA-R/LRP1 receptor complex (31, 33); however, there are important differences. First, S-PrP neutralized the response of macrophages to agonists that activate NOD1 and NOD2, whereas EI-tPA amplified these responses (33). Furthermore, EI-tPA was ineffective at neutralizing the effects of LPS on quiescent pMacs (33), whereas S-PrP was effective, suggesting that S-PrP may require a lower cell-surface abundance of NMDA-R in target cells. S-PrP is capable of targeting an expanded continuum of PRRs compared with previously studied anti-inflammatory proteins that engage the NMDA-R/LRP1 system. Understanding the unique qualities of S-PrP, which allow it to function as an inhibitor of NOD1 and NOD2, in addition to TLRs, is also important. The activity of S-PrP as an inhibitor of NOD2 is particularly intriguing because mutations in NOD2 have been associated with susceptibility to Crohn's Disease (56, 57).

Results obtained with BMDMs harvested from Prnp^{-/-} mice demonstrated that the NMDA-R/LRP1 complex does not require membrane-anchored PrP^{C} as a co-receptor to trigger cell-signaling in response to S-PrP. Experiments with the LRP1 antagonist, RAP, and with LRP1-deficient BMDMs from mLrp1^{-/-} mice suggested that LRP1 is important but not essential for mediating the anti-inflammatory activity of S-PrP. In the absence of LRP1, S-PrP was still active as an inhibitor of LPS-induced cytokine expression and IκBα phosphorylation; however, higher concentrations of S-PrP were required. These results are consistent with a model in which LRP1 sequesters S-PrP and delivers it to the NMDA-R to trigger cell-signaling. In the absence of LRP1, other macrophage cell surface macromolecules may sequester S-PrP for delivery to the NMDA-R. Alternatively, S-PrP may bind directly to the NMDA-R, albeit with lower avidity in the absence of LRP1, as has previously been described for tPA (29, 58, 59).

S-PrP is a recombinant protein; however, its structure is similar to that of a PrP^{C} derivative released from cells by ADAM10 (7). PrP^{C} serves as a substrate for other ADAMs, which generate solubilized PrP^{C} products differing in size and structure (7, 8). The results with POM2 suggest that the critical motif that interacts with the NMDA-R/LRP1 system to mediate anti-inflammatory cell-signaling is localized in the N-terminal unstructured region of PrP^{C} (26, 40). Other soluble PrP^{C} products that retain this epitope also may be active in regulating PPR activity. In a general sense, these studies demonstrate that solubilized PrP^{C} derivatives may be responsible for, or at least contribute to previously described anti-inflammatory activities of PrP^{C} (3, 20-22). Importantly, in many inflammatory cells, activators of innate immunity increase ADAM activity (60). Thus, release of soluble PrP^{C} derivatives may represent a feedback pathway by which innate immunity pathways are controlled.

By examining human blood bank plasma, the work by others (13, 14) demonstrating that EVs from blood carry PrP^{C} was confirmed. EVs are known to function in cell-cell communication, mainly through their ability to transfer biologically active cargo, including mRNAs, microRNAs, and proteins from a cell of origin to a target cell (10-12). The results presented in the present disclosure demonstrate that human plasma EVs may regulate innate immunity; however, the identified mechanism does not involve cargo transfer. Instead, the key event is the ability of a GPI-anchored membrane protein in EVs to function as a ligand and engage the NMDA-R/LRP1 receptor system in macrophages, which has known anti-inflammatory activity (30-33).

By performing experiments with PI-PLC, the immune regulatory factor in EVs is demonstrated to be GPI-anchored. PI-PLC completely eliminated the LPS-regulatory activity of human plasma EVs and this activity was recovered in the supernatant, as would be anticipated for a GPI-anchored protein that is known to be active in soluble form. The present disclosure provides studies that determined the GPI-anchored protein in human plasma EVs being PrP^{C} by neutralizing its activity with POM2. It is noteworthy that, from a battery of POM-specific monoclonal antibodies with defined epitopes (40), POM2 was the only antibody that blocked the activity of both S-PrP and EV-associated PrP^{C}. This result supports a model in which S-PrP and membrane-anchored PrP^{C} in EVs regulate PRRs by the equivalent mechanism.

Thus, the studies presented herein implicating the macrophage NMDA-R/LRP1 receptor complex as responsible for the anti-LPS/TLR4 activity of EV-associated PrP^{C} are novel because, although LRP1 functions as a receptor for numerous soluble ligands (27, 61, 62), LRP1 is not recognized as a receptor for EV-associated membrane proteins. The ability of EV-associated PrP^{C} to activate NMDA-R/LRP1-dependent cell-signaling in macrophages represents an entirely novel mechanism by which EVs may regulate immunity. The ability of LRP1 to function as an EV receptor merits further consideration. Because the results suggest that the NMDA-R/LRP1 receptor complex triggers cell-signaling in response to a membrane-anchored EV protein, it is important to test whether the NMDA-R/LRP1 receptor system triggers cell-signaling in response to plasma membrane proteins on neighboring cells.

Further, the results presented herein demonstrating that PI-PLC completely inactivates human plasma EVs as regulators of LPS suggest that PrP^{C} alone may be responsible for this EV activity. Although PI-PLC targets other GPI-anchored proteins, the conclusion that PrP^{C} is exclusively responsible for the anti-LPS/TLR4 activity of plasma EVs was supported by the results with POM2. Given the known heterogeneity in plasma EVs (10-12, 63-65), the activities demonstrated herein reflect a sub-population of human plasma EVs. It is important to determine whether the total abundance of plasma EV-associated PrP^{C} varies in diseases with chronic inflammatory components, and whether EV-associated PrP^{C} is active in the regulation of innate immunity in vivo.

LRP1 is known to function in phagocytosis of large particles (66) and in efferocytosis, as a receptor for apoptotic cells (67, 68). Based on these prior studies, it may suggest that LRP1 may function in EV targeting, binding, and cargo internalization, specifically for EVs with abundant PrP^{C}. The anti-inflammatory activity of EV PrP^{C} may reflect a more complicated set of mechanisms, beyond the ability to trigger NMDA-R/LRP1 receptor system-dependent cell-signaling.

SFKs were rapidly activated in macrophages treated with human plasma EVs. Furthermore, the effects of human plasma EVs on ERK1/2 phosphorylation, LPS-induced IκBα phosphorylation, and LPS-induced pro-inflammatory cytokine expression, in macrophages, were blocked by the SFK inhibitor, PP2, suggesting an essential role for SFKs. The present disclosure provides the first study implicating SFKs as critical upstream activators of the cell-signaling pathway triggered by any anti-inflammatory ligand for the NMDA-R/LRP1 receptor system in macrophages. SFKs have been implicated NMDA-R/LRP1 cell-signaling in neurons and neuron-like cells (26, 54). Similarly, SFKs are activated downstream of the NMDA-R in neurons treated with NMDA (69). Thus, although the outcome of NMDA-R-activated cell-signaling may depend on the cell type in which the NMDA-R is expressed, the upstream cell-signaling pathway may be at least partially conserved.

In summary, the present disclosure provides two states of PrP^{C} that are active in the regulation of innate immunity, shed PrP^{C} and PrP^{C} that is incorporated into EVs as a GPI-anchored protein. Both forms of PrP^{C} may contribute to the known anti-inflammatory activity of this gene product. The NMDA-R/LRP1 receptor system in macrophages serves as a receptor for both S-PrP and EV-associated PrP^{C}. These ligand-receptor interactions constitute a novel regulatory system in macrophage physiology.

### EXAMPLE 1

### Materials and Methods

### Proteins and Reagents

S-PrP (residues 23-231 from the structure of mouse PrP^{C}) was expressed and purified as previously described (26). In brief, S-PrP was expressed in E. coliBL21 as a His-tagged protein, which was recovered from inclusion bodies, denatured in guanidinium hydrochloride, purified by Ni²⁺-affinity chromatography, oxidized, and refolded out of denaturant. Thrombin was used to dissociate the N-terminal poly-His tail. The thrombin was then removed by ion exchange chromatography. S-PrP preparations were judged to be >98% pure by SDS-PAGE with silver staining and by LC-MS/MS analysis of tryptic peptides (26). All preparations were processed through high-capacity endotoxin removal columns (Pierce) and determined to be endotoxin-free using an endotoxin detection kit (Thermo Fisher Scientific).

Human enzymatically-inactive tPA (EI-tPA), which carries the S478A mutation and thus lacks catalytic activity, and a second mutation (R275E) so the protein remains in single-chain form, was from Molecular Innovations. α₂M was purified from human plasma, activated for binding to LRP1 by reaction with methylamine as previously described (39), and determined to be endotoxin-free. LPS serotype 055:B5 from E. coli was from Sigma-Aldrich. Lipoteichoic acid (LTA), ODN 1826, L18-MDP, and C12-iE-DAP were from InvivoGen. Imiquimod (IMQ) was from Frontier Scientific. Endotoxin-free, monomeric Receptor-associated Protein (RAP) was provided by Dr. Travis Stiles (Novoron Biosciences). Dizocilpine (MK801) was from Cayman Chemicals. Dextromethorphan hydrobromide (DXM) and the SFK inhibitor, PP2, were from Abcam. The monoclonal antibodies POM1, POM2, POM3, and POM19, which are directed against different epitopes in PrP^{C}, were purified as previously described (40). PI-PLC from B. cereus was purchased from Thermo Fisher Scientific.

### Animals

All animal experimental procedures were approved by the Institutional Animal Care and Use Committee of University of California San Diego. Wild-type (WT) C57BL/6J mice were obtained from Jackson Laboratory. To generate mice in which monocytes, macrophages, and neutrophils are LRP1 deficient (mLrp1^{-/-} mice), Lrp 1^{flox/flox} mice were bred with mice that express Cre recombinase under the control of the lysozyme-M promoter (LysM-Cre), in the C57BL/6J background, as previously described (41). For experiments with macrophages harvested from mLrp1^{-/-} mice, control cells were harvested from littermates that were LRP1^{flox/flox} but LysM-Cre-negative (mLrp1^{+/+} mice). Prnp^{-/-} mice were generously provided by Dr. Adriano Aguzzi (University Hospital of Zurich, Zurich, Switzerland).

### Cell culture

Bone marrow-derived macrophages (BMDMs) were harvested from 16-week-old male mice, as previously described (30). Briefly, bone marrow cells were flushed from mouse femurs and plated in non-tissue culture-treated dishes. Cells were cultured in DMEM/F-12 medium containing 10% fetal bovine serum (FBS) and 20% L929 cell-conditioned medium for 7 days. Non-adherent cells were eliminated. Adherent cells included >95% BMDMs, as determined by F4/80 and CD11b immunoreactivity.

Quiescent peritoneal macrophages (pMacs) were isolated from 16-week-old male C57BL/6J mice without thioglycollate elicitation and cultured as previously described (42). In brief, 5 ml of PBS (20 mM sodium phosphate, 150 mM NaCl, pH 7.4) with 3% FBS and 1× Gibco Antibiotic-Antimycotic (A/A) (Thermo Fisher Scientific) were injected into the peritoneal space with a 25-gauge needle. The solution was massaged from the abdominal surface and then harvested using the same needle. The procedure was repeated three times. Isolates that contained visible red blood cells were excluded. The remaining isolates were subjected to centrifugation at 800×g for 5 min, suspended in DMEM/F12 supplemented with 10% FBS and 1×A/A, and then plated at 2×10⁶ cells/well in tissue-culture treated 6-well plates. The cells were washed extensively 2 h after plating and maintained in culture for 48 hours before conducting experiments.

Cell viability

BMDMs were transferred to serum-free medium (SFM) for 30 min and then treated with S-PrP (40 or 120 nM) or vehicle for 6 h. The BMDMs were harvested and stained with 7-aminoactinomycin D (7-AAD), using the APC Annexin V Apoptosis Detection Kit (BioLegend), following the manufacturer's instructions. Apoptotic cells were detected by flow cytometry using a BD FACSCanto II (BD Biosciences). Data were analyzed with FlowJo Software version 10.7.1 (BD Biosciences).

Cell signaling

Cells were transferred to SFM for 30 min and then treated with various proteins and reagents, alone or simultaneously as noted, including: LPS (0.1 μg/ml); LTA (1.0 μg/mL); ODN 1826 (1 μM); IMQ (3 μg/ml); C12-iE-DAP (1 μg/mL); L18-MDP (0.1 μg/mL); S-PrP (20-120 nM); EI-tPA (12 nM); activated α₂M (10 nM); EVs (1.0-4.0 μg/mL); POM1, POM2, POM3, POM19, mouse IgG (10 μg/mL); or vehicle (PBS).

Cells were rinsed with ice-cold PBS and proteins were extracted in RIPA buffer (20 mM sodium phosphate, 150 mM NaCl, pH 7.4, 1% Triton X-100, 0.5% sodium deoxycholate, 0.1% SDS) supplemented with protease and phosphatase inhibitors (Thermo Fisher Scientific). Equal amounts of protein, as determined using the detergent-compatible (DC) Protein Assay (Bio-Rad), were subjected to 10% SDS-PAGE and electro-transferred to polyvinylidene fluoride membranes. The membranes were blocked with 5% nonfat dried milk and then incubated with primary antibodies from Cell Signaling Technology that recognize: phospho-ERK1/2, total ERK1/2, phospho-IκBα, IκBα, phospho-Tyr-416 in SFKs (the activation epitope), total SFKs, and β-actin. The membranes were washed and incubated with horseradish peroxidase-conjugated secondary antibody (Jackson ImmunoResearch). Immunoblots were developed using Radiance, Radiance Q, and Radiance Plus chemiluminescent substrates (Azure Biosystems) and imaged using the Azure Biosystems c300 digital system. The presented results are representative of at least three independent experiments.

### RT-qPCR

Cells were transferred to SFM for 30 min and then treated with various proteins and reagents for 6 h. RNA was isolated using the NucleoSpin RNA kit (Macherey-Nagel) and reverse-transcribed using the iScript cDNA synthesis kit (Bio-Rad). qPCR was performed using TaqMan gene expression products (Thermo Fisher Scientific). The relative change in mRNA expression was calculated using the 24ACT method with GAPDH mRNA as an internal normalizer. All results are presented as the fold-increase in mRNA expression relative to a specified control, in which cells were typically not treated with LPS or other reagents.

### LPS Challenge In Vivo

Male C57BL/6J mice (16-20 weeks old, ˜25 grams) were injected IP with 9 mg/kg LPS. The LD₅₀ for the specific LPS lot was pre-determined in our laboratory, as previously described by us (31) and was 6 mg/kg. One hour later, mice were treated by IV injection with S-PrP (2.5 μg/g body weight) or with PBS. Animals were monitored and scored at 3 h intervals using the Murine Sepsis Score (MSS), as described by Shrum et al. (43). In brief, the following variables were scored from 0-4: appearance, level of consciousness, activity, responses to auditory stimuli, eye function, respiration rate, and respiration quality. Mice were considered moribund and euthanized if the MSS was ≥21. Investigators were blinded to treatment groups. Survival was plotted in Kaplan-Meier curves.

### Isolation of Plasma EVs by Sequential Ultracentrifugation

Outdated human fresh frozen plasma (FFP) was obtained from the UCSD Transfusion Medicine service and studied without patient identifiers. The work presented herein was approved by the UCSD IRB for Human Investigation. FFP was subjected to centrifugation at 5,000×g for 10 min at 4° C. to ensure removal of platelets and cellular debris. The supernatant was collected and larger EVs were precipitated by ultracentrifugation (UC) for 2 h at 20,000×g at 4° C. (Avanti J Ultracentrifuge, Beckman Coulter). The supernatant was collected and subjected to UC at 100,000×g for 18 h at 4° C. The pellet was re-suspended in PBS, sterile-filtered using 0.22 μM syringe filters (EMD Millipore), and washed by UC at 100,000×g for 2 h at 4° C. (Opti-Max E, MLS-50 swinging-bucket rotor, Beckman Coulter). The EV-enriched pellet was re-suspended in PBS for analysis and experiments. The protein content of final EV preparations was determined by DC assay.

### Characterization of EVs

NTA was performed using a NanoSight NS300 instrument equipped with a 405 nm laser (Malvern). EV samples were passed through a fluidics flow chamber at a constant flow rate using a syringe pump at room temperature. Each sample was measured in duplicate at a camera setting of 11 with an acquisition time of 30 sec and detection threshold setting of 3. Data were captured and analyzed with NTA software, version 2.3 (Malvern Panalytical).

EV preparations were subjected to immunoblot analysis with antibodies that detect PrP^{C} (Abcam), Flotillin (BD Biosciences), Tsg101 (Abcam), and GM130 (BD Biosciences). For transmission electron microscopy (TEM) studies, isolated EVs were adsorbed to formvar/carbon-coated 100-mesh copper grids for 10 min, washed with water, and negatively stained with 2% uranyl acetate aqueous solution for 1 min. Grids were viewed using a JEOL 1200EX II TEM and photographed using a Gatan digital camera.

### Treatment of EVs with PI-PLC

PI-PLC releases GPI-anchored proteins from plasma membranes and thus, may be used to identify proteins that are anchored to plasma membranes by this type of linkage (37, 38). Equal amounts of EVs were treated with PI-PLC (0.1 units/mg EV protein) or with vehicle for 1 h at 4° C. with constant agitation. Samples were subjected to UC at 100,000×g for 2 h at 4° C. Supernatants, containing released proteins, were separated and retained for analysis. EV-containing pellets were washed once, re-suspended in PBS, and also retained for analysis.

### Statistics

Statistical analysis was performed using GraphPad Prism 9.0 (GraphPad Software). All results are expressed as the mean±SEM. When “n” values are reported, each replicate was performed using a different macrophage preparation or, when relevant, an EV preparation isolated from a different human plasma sample. Data were analyzed by one-way ANOVA followed by post-hoc Tukey's multiple comparison test. Kaplan-Meier survival curves were analyzed using the Mantel-Cox test. P-values of *P<0.05, **P<0.01, ***P<0.001, ****P<0.0001 were considered statistically significant.

### EXAMPLE 2

### S-PrP Neutralizes the Activity of LPS in Macrophages and In Vivo in Mice

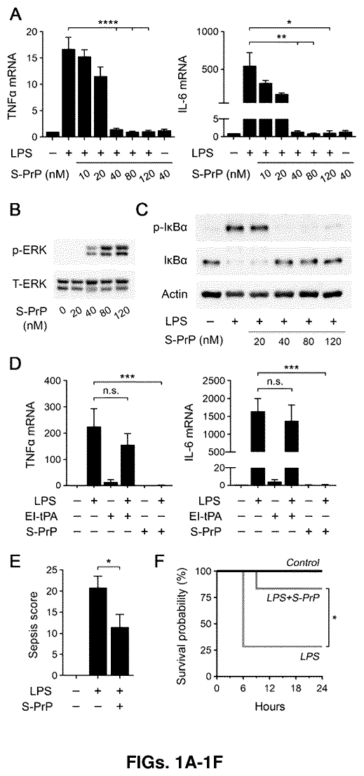

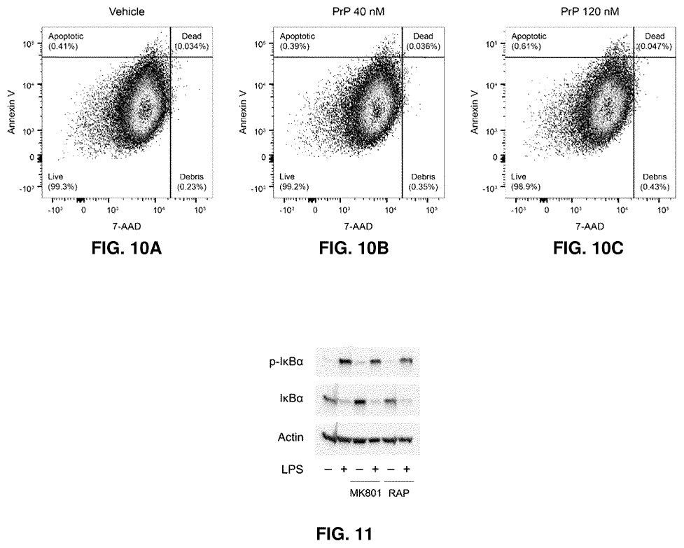

BMDMs were harvested from WT C57BL/6J mice and treated with 0.1 μg/mL LPS for 6 h in the presence of increasing concentrations of S-PrP. In the absence of S-PrP, LPS significantly increased expression of the mRNAs encoding TNFα and IL-6, as anticipated. S-PrP, at concentrations of 40 nM or higher, blocked the effects of LPS on expression of TNFα and IL-6 (FIG. 1A). In the absence of LPS, 40 nM S-PrP did not significantly regulate expression of TNFα or IL-6. Furthermore, in the absence of LPS, S-PrP (40 nM and 120 nM) did not affect BMDM viability (FIGS. 10A-10C).

To test whether S-PrP directly activates cell-signaling in BMDMs, in the absence of LPS, cells were treated with increasing concentrations of S-PrP for 1 h and then studied ERK1/2 phosphorylation. FIG. 1B shows that S-PrP, at concentrations greater than or equal to 40 nM, activated ERK1/2. The concentrations of S-PrP that activated ERK1/2 matched those that were effective in neutralizing LPS-stimulated cytokine expression (shown in FIG. 1A).

When BMDMs were treated for 1 h with 0.1 μg/mL LPS, in the absence of S-PrP, IκBα was phosphorylated and the abundance of IκBα was decreased (FIG. 1C). These effects of LPS report activation of NFκB as a transcription factor, which is essential for expression of pro-inflammatory cytokines (44). In BMDMs treated simultaneously with LPS and 40-120 nM S-PrP, the effects of LPS on IκBαphosphorylation and abundance were blocked.

As a second model system to study the activity of S-PrP, we isolated macrophages from the peritoneal space of mice (pMacs) without eliciting or activating agents (33, 42). Compared with BMDMs, pMacs express lower levels of cell-surface NMDA-R and, as a result, are incapable of responding to the NMDA-R/LRP1 receptor system ligand, EI-tPA (33). The ability of S-PrP to neutralize the response to LPS was examined in pMacs and, as a control, EI-tPA was re-examined. FIG. 1D shows that 0.1 μg/mL LPS increased expression of the mRNAs encoding TNFα and IL-6 in pMacs. When pMacs were treated simultaneously with LPS and 12 nM EI-tPA, a concentration of EI-tPA that is fully effective in blocking LPS activity in BMDMs (31), cytokine mRNA expression in pMacs was not inhibited, confirming the earlier results (33). By contrast, 40 nM S-PrP completely blocked LPS-induced cytokine mRNA expression in pMacs. Thus, S-PrP is active as an anti-LPS/TLR4 agent in cells in which the NMDA-R/LRP1 ligand, EI-tPA, does not demonstrate efficacy.

To test the ability of S-PrP to inhibit TLR4 responses in vivo, C57BL/6J mice (˜25 gram) were treated by IV injection with 2.5 μg/g body weight S-PrP (n=6) or vehicle (n=7), 1 h after injecting LPS at 1.5× the LD₅₀. Animals were scored for toxicity at 3 h intervals, examining criteria that included level of consciousness, appearance, activity, response to auditory stimuli, respiration quality and rate (43). Mice that entered a moribund state were euthanized immediately. FIG. 1E shows that toxicity scores, determined at 6 h when these scores maximized in animals treated with LPS alone, were significantly decreased in mice treated with S-PrP. FIG. 1F shows that more than half of the mice treated with LPS alone required euthanasia due to the degree of toxicity. A single dose of S-PrP significantly improved survival.

### EXAMPLE 3

### S-PrP Targets a Large Continuum of PRRs in Innate Immunity

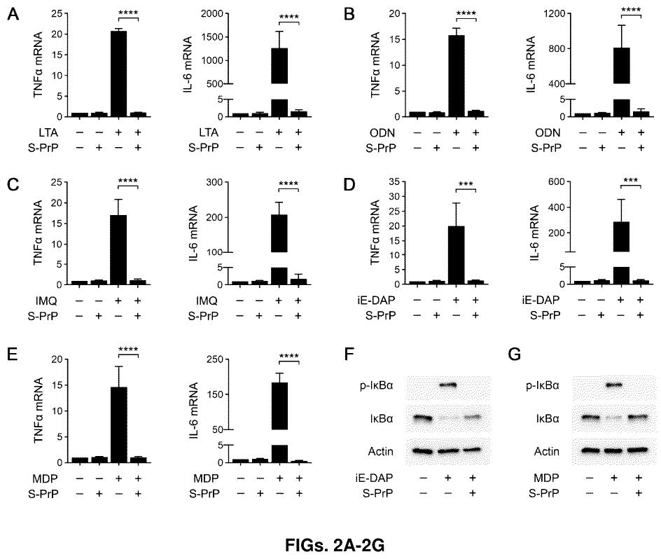

LTA is a selective TLR2 agonist, produced by gram-positive bacteria (45, 46). BMDMs treated with 1.0 μg/mL LTA for 6 h demonstrated increased expression of the mRNAs encoding TNFα and IL-6, as anticipated (FIG. 2A). S-PrP (40 nM) neutralized the effects of LTA on expression of TNFα and IL-6.

ODN 1826 is a TLR9 agonist (47, 48). ODN 1826 (1.0 μM) significantly increased expression of TNFα and IL-6 mRNA in BMDMs (FIG. 2B). S-PrP blocked the effects of ODN 1826 on expression of TNFα and IL-6. Equivalent results were obtained when we studied the TLR7 agonist, IMQ. IMQ (3.0 μg/mL) induced expression of TNFα and IL-6 mRNA in BMDMs and the response was blocked by S-PrP (FIG. 2C). These results support the conclusion that S-PrP is a generalized inhibitor of macrophage responses elicited by TLRs.

NOD1 and NOD2 are intracellular PRRs (49, 50). Unlike TLRs, the responses elicited by NOD1 and NOD2 agonists in BMDMs are not neutralized by EI-tPA and may in fact be amplified (33). BMDMs were treated with the NOD1 agonist, C12-iE-DAP (1 μg/mL), or the NOD2 agonist, L18-muramyl dipeptide (MDP) (0.1 μg/mL), for 6 h. FIGS. 2D and 2E show that both agents increased expression of the mRNAs encoding TNFα and IL-6. When BMDMs were treated with C12-iE-DAP and 40 nM S-PrP simultaneously, the effects of C12-iE-DAP on cytokine mRNA expression were neutralized. Similarly, 40 nM S-PrP blocked cytokine mRNA expression in response to MDP.

IκBα was phosphorylated and the abundance of IκBα was decreased in BMDMs treated with C12-iE-DAP (1 μg/mL) for 1 h (FIG. 2F). These effects of C12-iE-DAP were blocked by S-PrP (40 nM). S-PrP also inhibited IκBαphosphorylation in response to MDP (0.1 μg/mL) (FIG. 2G). Collectively, these results show that S-PrP neutralizes responses elicited by PRRs in addition to TLRs and thus, may target a broader continuum of PRRs, compared with EI-tPA (33).

### EXAMPLE 4

### S-PrP Inhibits PRRs by Interacting with the Macrophage NMDA-R/LRP1 Receptor System

The ability of PrP^{C}-specific monoclonal antibodies with defined epitopes to neutralize the effects of S-PrP on the LPS response were tested in BMDMs. POM1 and POM19 recognize epitopes in the C-terminal globular region of PrP^{C}, whereas POM2 recognizes the tandem octarepeats in the N-terminal unstructured region of PrP^{C} (40). POM3 recognizes an epitope C-terminal to the POM2 epitope, near the center of PrP^{C} (40).

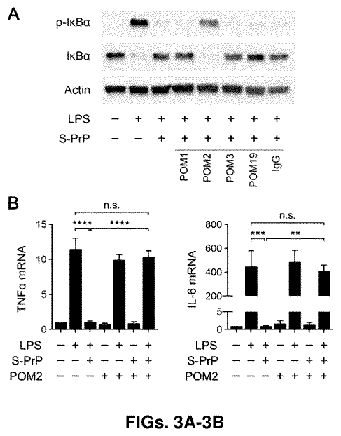

BMDMs were treated with 0.1 μg/mL LPS and 40 nm S-PrP in the presence of each antibody (10 μg/ml) for 1 h. POM2 completely blocked the ability of S-PrP to inhibit LPS-induced IκBα phosphorylation (FIG. 3A). The other antibodies were without effect, as was non-specific IgG. In cytokine expression studies, POM2 blocked the ability of S-PrP to inhibit expression of TNFα and IL-6 in BMDMs treated with 0.1 μg/mL LPS (FIG. 3B). POM2 did not significantly affect cytokine expression in BMDMs in the absence of S-PrP. These results implicate a site in the N-terminal unstructured region of S-PrP as critical for the anti-LPS/TLR4 activity of S-PrP.

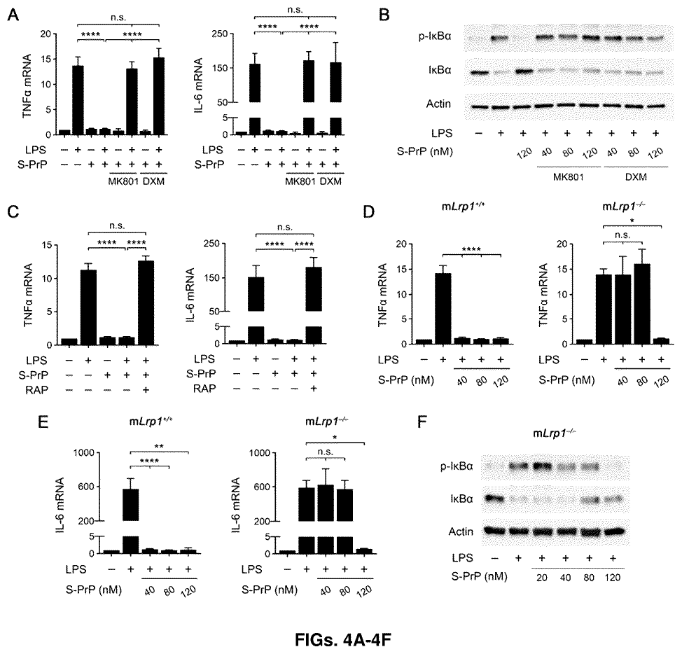

The NMDA-R is expressed by macrophages and essential for the anti-inflammatory activity of EI-tPA and activated α₂M (31). Because the NMDA-R mediates cell-signaling in response to S-PrP in neuron-like cells and Schwann cells (26), whether the NMDA-R is required for the anti-inflammatory activity of S-PrP in macrophages was tested. BMDMs were treated with the non-competitive NMDA-R antagonists, MK801 (1.0 μM) or DXM (10 μM). Both reagents completely blocked the ability of 40 nM S-PrP to neutralize inflammatory cytokine mRNA expression in response to 0.1 μg/mL LPS (FIG. 4A). MK801 and DXM also blocked the ability of 40 nM S-PrP to inhibit LPS-induced IκBα phosphorylation (FIG. 4B). The effects of the NMDA-R antagonists were not overcome by increasing the concentration of S-PrP to 120 nM. In control experiments, MK801 and DXM did not alter the effects of LPS on IκBα phosphorylation or total abundance in the absence of S-PrP, as anticipated (FIG. 11).

Next, the activity of LRP1 as a mediator of the response to S-PrP in BMDMs was also studied. BMDMs were treated with 0.1 μg/mL LPS and 40 nM S-PrP, in the presence and absence of RAP, a protein antagonist of ligand-binding to LRP1 and other members of the LDL receptor family (27, 51). RAP (150 nM) blocked the ability of 40 nM S-PrP to neutralize LPS-induced expression of the mRNAs encoding TNFα and IL-6 (FIG. 4C), suggesting a role for LRP1.

To confirm the role of LRP1, BMDMs were isolated from mLrp1^{-/-} mice. LRP1 protein is undetectable in macrophages from these mice (30, 41). Control LRP1-expressing BMDMs were harvested from mLrp1^{+/+} mice. LPS (0.1 μg/mL) increased expression of the mRNAs encoding TNFα and IL-6 in LRP1-expressing and -deficient BMDMs similarly (FIGS. 4D & 4E). S-PrP (40-120 nM) blocked LPS-induced cytokine mRNA expression in LRP1-expressing BMDMs isolated from mLrp1^{+/+} mice. By contrast, S-PrP (40-80 nM) was ineffective at inhibiting LPS-induced cytokine mRNA expression in LRP1-deficient BMDMs. The activity of S-PrP was restored in BMDMs from mLRP1^{-/-} mice when the S-PrP concentration was increased to 120 nM.

FIG. 4F shows that the concentration of S-PrP required to neutralize the effects of LPS on IκBα phosphorylation was increased from 40 nM, which was effective in LRP1-expressing BMDMs (see FIG. 1C), to 120 nM in LRP1-deficient BMDMs from mLRP1^{-/-} mice. These results suggest that LRP1 deficiency in BMDMs does not eliminate the anti-LPS/TLR4 activity of S-PrP, but instead, increases the concentration of S-PrP required for efficacy.

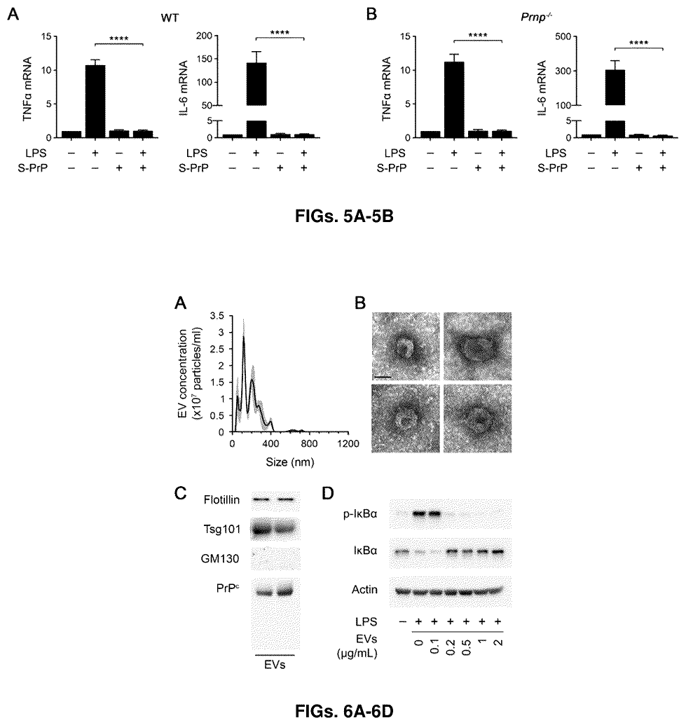

Mattei et al (35) reported that membrane-anchored PrP^{C} is required for tPA-activated cell-signaling in neurons. To test whether membrane-anchored PrP^{C} is required for macrophages to respond to S-PrP, BMDMs isolated from mice with global deletion of Prnp, the gene encoding PrP^{C} (52) were studied. S-PrP (40 nM) blocked the effects of LPS on expression of the mRNAs encoding TNFα and IL-6 equivalently in WT BMDMs (FIG. 5A) and PrP^{C}-deficient BMDMs (FIG. 5B). Thus, macrophages do not appear to require membrane-anchored PrP^{C} to respond to S-PrP as an anti-LPS/TLR4 agent.

### EXAMPLE 5

### Human Plasma EVs Demonstrate Anti-Inflammatory Activity by Engaging the NMDA-R/LRP1 Receptor System

EVs were isolated from human blood bank plasma by sequential ultracentrifugation (UC), as previously described (13, 53). Nanoparticle Tracking Analysis (NTA) demonstrated particles of variable size, ranging from 50-400 nm (FIG. 6A). The NTA results indicate that the isolated EVs were heterogeneous.

Human plasma EVs were negatively stained and examined by TEM. FIG. 6B shows representative examples of EVs present in the UC EV preparations from human plasma. The exosome biomarker, flotillin, a lipid raft associated protein, and the cytosolic marker, Tumor Susceptibility 101 (Tsg101), were identified in two representative plasma UC EV preparations, together with PrP^{C} (FIG. 6C). The golgi matrix protein, GM130, was absent from human plasma EVs.

Because human plasma EVs carry PrP^{C}, it was tested whether these EVs replicate the activity of S-PrP in experiments with cultured macrophages. BMDMs were treated with LPS (0.1 μg/mL) and increasing amounts of human plasma EVs for 1 h. IκBα phosphorylation and abundance were examined. Human plasma EVs blocked the effects of LPS on IκBα phosphorylation and abundance in an EV concentration-dependent manner (FIG. 6D). Complete inhibition of the LPS response was observed when the amount of EV-associated protein added to the cultures was equal to or exceeded 0.2 μg/mL.

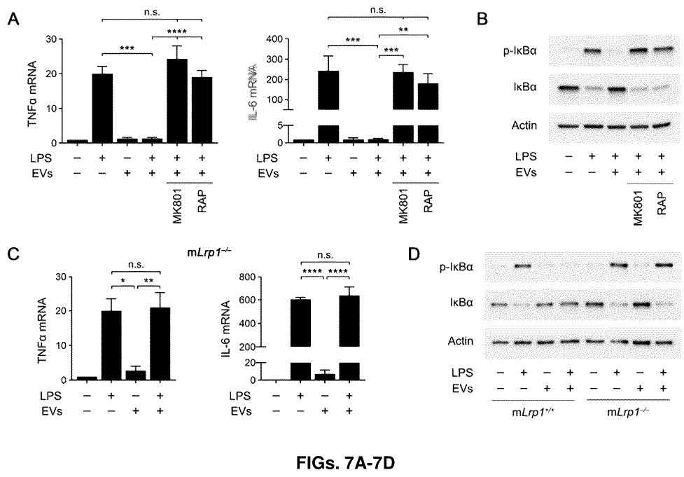

Next, the ability of human plasma EVs to regulate cytokine mRNA expression were tested in BMDMs treated with LPS. FIG. 7A shows that human plasma EVs (1.0 μg/mL) blocked expression of TNFα and IL-6 mRNA in BMDMs treated with 0.1 μg/mL LPS for 6 h. Plasma EVs did not regulate expression of TNFα or IL-6 in the absence of LPS. The effects of the EVs on LPS-induced cytokine expression were neutralized by the NMDA-R antagonist, MK801, suggesting an essential role for the NMDA-R. Furthermore, the effects of EVs on LPS-induced cytokine expression were blocked by RAP, suggesting a role for LRP1. MK801 and RAP also blocked the ability of human plasma EVs to inhibit IκBαphosphorylation and the accompanying decrease in IκBα abundance in LPS-treated BMDMs (FIG. 7B).

To confirm that macrophage LRP1 mediates the anti-LPS/TLR4 activity of human plasma EVs, LRP1-deficient BMDMs isolated from mLrp1^{-/-} mice were also studied. LPS (0.1 μg/mL) increased expression of the mRNAs encoding TNFα and IL-6 in LRP1-deficient BMDMs (FIG. 7C). Human plasma EVs (1.0 μg/mL) failed to inhibit this response. Similarly, human plasma EVs failed to inhibit the effects of LPS on IκBα phosphorylation in LRP1-deficient BMDMs (FIG. 7D). These results confirm that macrophage LRP1 mediates the anti-LPS/TLR4 activity of human plasma EVs.

### EXAMPLE 6

### The Anti-LPS/TLR4 Activity of Human Plasma EVs Requires EV-Associated, GPI-Anchored PrP^{C}

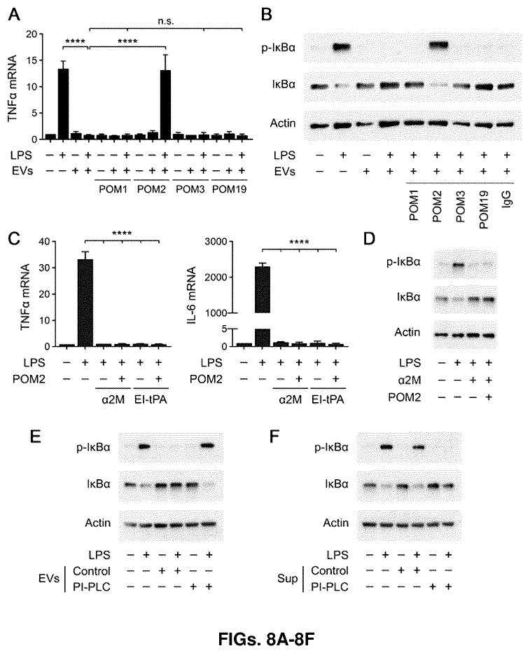

To test whether EV-associated PrP^{C} is responsible for the effects of human plasma EVs on LPS-induced TNFα expression, LPS (0.1 μg/mL) and EVs (1.0 μg/mL) were added to BMDM cultures in the presence of the PrP^{C}-specific antibodies: POM1, POM2, POM3, or POM19 (each at 10 μg/ml). POM2 completely neutralized the anti-LPS activity of the EVs, restoring TNFα expression to the level observed in cells treated with LPS alone (FIG. 8A). The other antibodies were entirely ineffective. This result is important showing POM2 is the only PrP^{C}-targeting antibody that blocked the anti-LPS/TLR4 activity of recombinant S-PrP. In control studies, the antibodies examined did not independently regulate TNFα expression in the absence of LPS.

Next, the activity of the POM antibodies was examined in IκBα phosphorylation experiments. FIG. 8B shows that POM2 completely blocked the ability of human plasma EVs to inhibit IκBα phosphorylation and the accompanying decrease in the IκBα abundance in BMDMs treated with LPS (0.1 μg/mL). The PrP^{C}-specific antibodies, POM1, POM3, and POM19, and non-specific IgG were without effect.

These results with BMDMs isolated from Prnp^{-/-} mice suggested that macrophage-associated PrP^{C} is not required to mediate the anti-LPS/TLR4 activity of S-PrP. To confirm that POM2 blocks the anti-inflammatory activity of human plasma EVs by targeting EV-associated PrP^{C} and not macrophage-associated PrP^{C}, the activity of POM2 was studied in experiments with two distinct proteins that block LPS/TLR4 responses in macrophages by engaging the NMDA-R/LRP1 receptor system: α²M and EI-tPA. FIG. 8C shows that 10 μg/mL POM2 had no effect on the ability of 12 nM EI-tPA or 10 nM α₂M to inhibit expression of the mRNAs encoding TNFα or IL6 in LPS-treated BMDMs. Similarly, POM2 did not interfere with the ability of 10 nM α₂M to inhibit LPS-induced IκBα phosphorylation (FIG. 8D). These results support the conclusion that POM2 inhibits the anti-LPS/TLR4 activity of human plasma EVs by targeting EV-associated PrP^{C} and not macrophage PrP^{C}.

To confirm the role EV-associated PrP^{C} as the principal factor responsible for the anti-LPS/TLR4 activity of human plasma EVs and to determine the state of PrP^{C} in plasma EVs, human plasma EVs were treated with PI-PLC, which cleaves and releases GPI-anchored proteins from plasma membranes (37, 38). Following PI-PLC treatment, EVs were washed by UC at 100,000×g to separate the EVs from solution-phase components. Control EVs were not PI-PLC-treated but still subjected to the same washing protocol. FIG. 8E shows that, unlike control EVs, PI-PLC-treated EVs were totally inactive as inhibitors of LPS-induced IκBα phosphorylation in BMDMs. In control experiments, PI-PLC-treated EVs did not independently induce IκBα phosphorylation.

Next, human plasma EVs were treated with PI-PLC or vehicle and subjected to washing by UC. Instead of studying the EV-containing pellet, the supernatants were studied. Supernatants that were harvested from control EVs, which were not PI-PLC-treated, were inactive at inhibiting LPS-induced IκBα phosphorylation (FIG. 8F). By contrast, supernatants that were harvested from PI-PLC-treated EVs were active, blocking LPS-induced IκBα phosphorylation and the accompanying decrease in abundance of IκBα. Thus, PI-PLC treatment dissociates the anti-LPS/TLR4 activity from human plasma EVs without destroying this activity. Collectively, the POM2 and PI-PLC studies strongly suggest that EV-associated PrP^{C} is responsible for the anti-LPS/TLR4 activity of human plasma EVs and that the bioactive form of PrP^{C} in EVs is GPI-anchored.

### EXAMPLE 7

### SFKs are Required for the Anti-LPS/TLR4 Activity of Human Plasma EVs

In neuron-like cells, SFKs are important upstream mediators of cell-signaling responses elicited by S-PrP and other ligands that engage the NMDA-R/LRP1 receptor system (26, 54). SFKs also have been implicated in anti-inflammatory responses mediated by membrane-anchored PrP^{C} (23). Thus, studies were performed to determine whether SFKs function in the pathway by which human plasma EV-associated PrP^{C} regulates macrophage physiology.

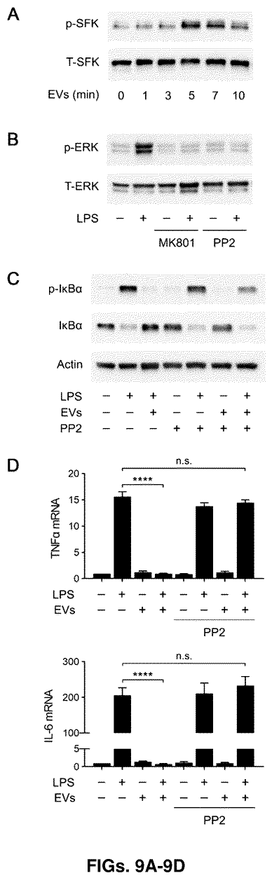

FIG. 9A shows that human plasma EVs rapidly activated SFKs in BMDMs. Phospho-SFK-Tyr-416 was observed within 5 min of adding 4 μg/mL EVs. FIG. 9B shows that ERK1/2 was activated in BMDMs treated with human plasma EVs for 1 h. This response was entirely blocked by the SFK inhibitor, PP2 (1 μM). Similarly, PP2 blocked the ability of human plasma EVs to inhibit IκBαphosphorylation in response to LPS (FIG. 9C). FIG. 9D shows that PP2 blocked the ability of plasma EVs to inhibit cytokine expression in response to LPS. These results implicate SFKs as upstream mediators of the response to EV-associated PrP^{C}, which neutralizes the activity of LPS in macrophages.

### REFERENCES

1. Taylor, D. R., and N. M. Hooper. 2006. The prion protein and lipid rafts. Mol. Membr. Biol. 23: 89-99.
2. Pan, K. M., M. Baldwin, J. Nguyen, M. Gasset, A. Serban, D. Groth, I. Mehlhorn, Z. Huang, R. J. Fletterick, F. E. Cohen, and S. B. Prusiner. 1993. Conversion of a-helices into β-sheets features in the formation of the scrapie prion proteins. Proc. Natl. Acad. Sci. U. S. A. 90: 10962-10966.
3. Bakkebø, M. K., S. Mouillet-Richard, A. Espenes, W. Goldmann, J. Tatzelt, and M. A. Tranulis. 2015. The cellular prion protein: A player in immunological quiescence. Front. Immunol. 6: 450.
4. Almeida, C. J. G. de, L. B. Chiarini, J. P. da Silva, P. M. R. e Silva, M. A. Martins, R. Linden, C. J. G. de Almeida, L. B. Chiarini, J. P. da Silva, P. M. R. e Silva, M. A. Martins, and R. Linden. 2005. The cellular prion protein modulates phagocytosis and inflammatory response. J. Leukoc. Biol. 77: 238-246.
5. Dürig, J., A. Giese, W. Schulz-Schaeffer, C. Rosenthal, U. Schmücker, J. Bieschke, U. Dührsen, and H. A. Kretzschmar. 2000. Differential constitutive and activation-dependent expression of prion protein in human peripheral blood leucocytes. Br. J. Haematol. 108: 488-495.
6. James Haddon, D., M. R. Hughes, F. Antignano, D. Westaway, N. R. Cashman, and K. M. McNagny. 2009. Prion protein expression and release by mast cells after activation. J. Infect. Dis. 200: 827-831.
7. Taylor, D. R., E. T. Parkin, S. L. Cocklin, J. R. Ault, A. E. Aschcroft, A. J. Turner, and N. M. Hooper. 2009. Role of ADAMs in the ectodomain shedding and conformational conversion of the prion protein. J. Biol. Chem. 284: 22590-22600.
8. McDonald, A. J., J. P. Dibble, E. G. B. Evans, and G. L. Millhauser. 2014. A new paradigm for enzymatic control of α-cleavage and β-cleavage of the prion protein. J. Biol. Chem. 289: 803-813.
9. Liang, J., W. Wang, D. Sorensen, S. Medina, S. Ilchenko, J. Kiselar, W. K. Surewicz, S. A. Booth, and Q. Kong. 2012. Cellular prion protein regulates its own α-cleavage through ADAM8 in skeletal muscle. J. Biol. Chem. 287: 16510-16520.
10. Théry, C., L. Zitvogel, and S. Amigorena. 2002. Exosomes: Composition, biogenesis and function. Nat. Rev. Immunol. 2: 569-579.
11. Raposo, G., and W. Stoorvogel. 2013. Extracellular vesicles: Exosomes, microvesicles, and friends. J. Cell Biol. 200: 373-383.
12. Maas, S. L. N., X. O. Breakefield, and A. M. Weaver. 2017. Extracellular Vesicles: Unique Intercellular Delivery Vehicles. Trends Cell Biol. 27: 172-188.
13. Ritchie, A. J., D. M. Crawford, D. J. P. Ferguson, J. Burthem, and D. J. Roberts. 2013. Normal prion protein is expressed on exosomes isolated from human plasma. Br. J. Haematol. 163: 678-680.
14. Robertson, C., S. A. Booth, D. R. Beniac, M. B. Coulthart, T. F. Booth, and A. McNicol. 2006. Cellular prion protein is released on exosomes from activated platelets. Blood 107: 3907-3911.
15. Fevrier, B., D. Vilette, F. Archer, D. Loew, W. Faigle, M. Vidal, H. Laude, and G. Raposo. 2004. Cells release prions in association with exosomes. Proc. Natl. Acad. Sci. U. S. A. 101: 9683-9688.
16. Vella, L. J., D. L. V. Greenwood, R. Cappai, J. P. Y. Scheerlinck, and A. F. Hill. 2008. Enrichment of prion protein in exosomes derived from ovine cerebral spinal fluid. Vet. Immunol. Immunopathol. 124: 385-393.
17. Falker, C., A. Hartmann, I. Guett, F. Dohler, H. Altmeppen, C. Betzel, R. Schubert, D. Thurm, F. Wegwitz, P. Joshi, C. Verderio, S. Krasemann, and M. Glatzel. 2016. Exosomal cellular prion protein drives fibrillization of amyloid beta and counteracts amyloid beta-mediated neurotoxicity. J. Neurochem. 137: 88-100.
18. Hartmann, A., C. Muth, O. Dabrowski, S. Krasemann, and M. Glatzel. 2017. Exosomes and the prion protein: More than one truth. Front. Neurosci. 11: 194.
19. Abels, E. R., and X. O. Breakefield. 2016. Introduction to Extracellular Vesicles: Biogenesis, RNA Cargo Selection, Content, Release, and Uptake. Cell. Mol. Neurobiol. 36: 301-312.
20. Onodera, T., A. Sakudo, H. Tsubone, and S. Itohara. 2014. Review of studies that have used knockout mice to assess normal function of prion protein under immunological or pathophysiological stress. Microbiol. Immunol. 58: 361-374.
21. Tsutsui, S., J. N. Hahn, T. A. Johnson, Z. Ali, and F. R. Jirik. 2008. Absence of the cellular prion protein exacerbates and prolongs neuroinflammation in experimental autoimmune encephalomyelitis. Am. J. Pathol. 173: 1029-1041.
22. Gourdain, P., C. Ballerini, A. B. Nicot, and C. Carnaud. 2012. Exacerbation of experimental autoimmune encephalomyelitis in prion protein (PrPc)-null mice: Evidence for a critical role of the central nervous system. J. Neuroinflammation 9: 524.
23. Chida, J., H. Hara, K. Uchiyama, E. Takahashi, H. Miyata, H. Kosako, Y. Tomioka, T. Ito, H. Horiuchi, H. Matsuda, H. Kido, and S. Sakaguchi. 2020. Prion protein signaling induces M2 macrophage polarization and protects from lethal influenza infection in mice. PLOS Pathog. 16: e1008823.
24. Liu, J., D. Zhao, C. Liu, T. Ding, L. Yang, X. Yin, and X. Zhou. 2015. Prion Protein Participates in the Protection of Mice from Lipopolysaccharide Infection by Regulating the Inflammatory Process. J. Mol. Neurosci. 55: 279-287.
25. Martin, G. R., C. M. Keenan, K. A. Sharkey, and F. R. Jirik. 2011. Endogenous prion protein attenuates experimentally induced colitis. Am. J. Pathol. 179: 2290-2301.
26. Mantuano, E., P. Azmoon, M. A. Banki, M. S. Lam, C. J. Sigurdson, and S. L. Gonias. 2020. A soluble derivative of PrPC activates cell-signaling and regulates cell physiology through LRP1 and the NMDA receptor. J. Biol. Chem. 295: 14178-14188.
27. Strickland, D. K., S. L. Gonias, and W. S. Argraves. 2002. Diverse roles for the LDL receptor family. Trends Endocrinol. Metab. 13: 66-74.
28. Martin, A. M., C. Kuhlmann, S. Trossbach, S. Jaeger, E. Waldron, A. Roebroek, H. J. Luhmann, A. Laatsch, S. Weggen, V. Lessmann, and C. U. Pietrzik. 2008. The functional role of the second NPXY motif of the LRP1 β-chain in tissue-type plasminogen activator-mediated activation of N-methyl-D-aspartate receptors. J. Biol. Chem. 283: 12004-12013.
29. Mantuano, E., M. S. Lam, and S. L. Gonias. 2013. LRP1 assembles unique co-receptor systems to initiate cell signaling in response to tissue-type plasminogen activator and myelin-associated glycoprotein. J. Biol. Chem. 288: 34009-34018.
30. Mantuano, E., C. Brifault, M. S. Lam, P. Azmoon, A. S. Gilder, and S. L. Gonias. 2016. LDL receptor-related protein-1 regulates NFκB and microRNA-155 in macrophages to control the inflammatory response. Proc. Natl. Acad. Sci. 113: 1369-1374.
31. Mantuano, E., P. Azmoon, C. Brifault, M. A. Banki, A. S. Gilder, W. M. Campana, and S. L. Gonias. 2017. Tissue-type Plasminogen Activator Regulates Macrophage Activation and Innate Immunity. Blood 130: 1364-1374.
32. Zalfa, C., P. Azmoon, E. Mantuano, and S. L. Gonias. 2019. Tissue-type plasminogen activator neutralizes LPS but not protease-activated receptor-mediated inflammatory responses to plasmin. J. Leukoc. Biol. 105: 729-740.
33. Das, L., P. Azmoon, M. A. Banki, E. Mantuano, and S. L. Gonias. 2019. Tissue-type plasminogen activator selectively inhibits multiple toll-like receptors in CSF-1differentiated macrophages. PLOS One 14: e0224738.
34. Parkyn, C. J., E. G. M. Vermeulen, R. C. Mootoosamy, C. Sunyach, C. Jacobsen, C. Oxvig, S. Moestrup, Q. Liu, G. Bu, A. Jen, and R. J. Morris. 2008. LRP1 controls biosynthetic and endocytic trafficking of neuronal prion protein. J. Cell Sci. 121: 773-783.
35. Mattei, V., V. Manganelli, S. Martellucci, A. Capozzi, E. Mantuano, A. Longo, A. Ferri, T. Garofalo, M. Sorice, and R. Misasi. 2020. A multimolecular signaling complex including PrPC and LRP1 is strictly dependent on lipid rafts and is essential for the function of tissue plasminogen activator. J. Neurochem. 152: 468-481.
36. Ellis, V., M. Daniels, R. Misra, and D. R. Brown. 2002. Plasminogen activation is stimulated by prion protein and regulated in a copper-dependent manner. Biochemistry 41: 6891-6896.
37. Hirose, S., J. J. Knez, and M. Edward Medof. 1995. Mammalian Glycosylphosphatidylinositol-Anchored Proteins and Intracellular Precursors. Methods Enzymol. 250: 582-614.
38. Griffith, O. H., and M. Ryan. 1999. Bacterial phosphatidylinositol-specific phospholipase C: Structure, function, and interaction with lipids. Biochim. Biophys. Acta-Mol. Cell Biol. Lipids 1441: 237-254.
39. Imber, M. J., and S. V Pizzo. 1981. Clearance and binding of two electrophoretic “fast” forms of human alpha 2-macroglobulin. J Biol Chem 256: 8134-8139.
40. Polymenidou, M., R. Moos, M. Scott, C. Sigurdson, Y. Shi, B. Yajima, I. Hafner-Bratkovič, R. Jerala, S. Hornemann, K. Wuthrich, A. Bellon, M. Vey, G. Garen, M. N. G. James, N. Kav, and A. Aguzzi. 2008. The POM Monoclonals: A Comprehensive Set of Antibodies to Non-Overlapping Prion Protein Epitopes. PLOS One 3: e3872.
41. Staudt, N. D., M. Jo, J. Hu, J. M. Bristow, D. P. Pizzo, A. Gaultier, S. R. VandenBerg, and S. L. Gonias. 2013. Myeloid cell receptor LRP1/CD91 regulates monocyte recruitment and angiogenesis in tumors. Cancer Res. 73: 3902-3912.
42. Zhang, X., R. Goncalves, and D. M. Mosser. 2008. The isolation and characterization of murine macrophages. Curr Protoc Immunol Chapter 14: Unit 14.1.
43. Shrum, B., R. V Anantha, S. X. Xu, M. Donnelly, S. M. M. Haeryfar, J. K. McCormick, and T. Mele. 2014. A robust scoring system to evaluate sepsis severity in an animal model. BMC Res. Notes 7: 1-11.
44. Tak, P. P., and G. S. Firestein. 2001. NF-κB: A key role in inflammatory diseases. J. Clin. Invest. 107: 7-11.
45. Schwandner, R., R. Dziarski, H. Wesche, M. Rothe, and C. J. Kirschning. 1999. Peptidoglycan- and lipoteichoic acid-induced cell activation is mediated by Toll-like receptor 2. J. Biol. Chem. 274: 17406-17409.
46. Schneewind, O., and D. Missiakas. 2014. Lipoteichoic acids, phosphate-containing polymers in the envelope of gram-positive bacteria. J Bacteriol 196: 1133-1142.
47. Vollmer, J., R. Weeratna, P. Payette, M. Jurk, C. Schetter, M. Laucht, T. Wader, S. Tluk, M. Liu, H. L. Davis, and A. M. Krieg. 2004. Characterization of three CpG oligodeoxynucleotide classes with distinct immunostimulatory activities. Eur J Immunol 34: 251-262.
48. Ballas, Z. K., A. M. Krieg, T. Warren, W. Rasmussen, H. L. Davis, M. Waldschmidt, and G. J. Weiner. 2001. Divergent Therapeutic and Immunologic Effects of Oligodeoxynucleotides with Distinct CpG Motifs. J. Immunol. 167: 4878-4886.
49. Strober, W., P. J. Murray, A. Kitani, and T. Watanabe. 2006. Signalling pathways and molecular interactions of NOD1 and NOD2. Nat Rev Immunol 6: 9-20.
50. Kim, Y. G., J. H. Park, M. H. Shaw, L. Franchi, N. Inohara, and G. Núñez. 2008. The cytosolic sensors Nod1 and Nod2 are critical for bacterial recognition and host defense after exposure to Toll-like receptor ligands. Immunity 28: 246-257.
51. Williams, S. E., J. D. Ashcom, W. S. Argraves, and D. K. Strickland. 1992. A novel mechanism for controlling the activity of alpha 2-macroglobulin receptor/low density lipoprotein receptor-related protein. Multiple regulatory sites for 39-kDa receptor-associated protein. J. Biol. Chem. 267: 9035-9040.
52. Nuvolone, M., M. Hermann, S. Sorce, G. Russo, C. Tiberi, P. Schwarz, E. Minikel, D. Sanoudou, P. Pelczar, and A. Aguzzi. 2016. Strictly co-isogenic C57BL/6J-Prnp−/− mice: A rigorous resource for prion science. J. Exp. Med. 213: 313-327.
53. Das, S., The Extracellular RNA Communication Consortium, K. M. Ansel, M. Bitzer, X. O. Breakefield, A. Charest, D. J. Galas, M. B. Gerstein, M. Gupta, A. Milosavljevic, M. T. McManus, T. Patel, R. L. Raffai, J. Rozowsky, M. E. Roth, J. A. Saugstad, K. Van Keuren-Jensen, A. M. Weaver, and L. C. Laurent. 2019. The Extracellular RNA Communication Consortium: Establishing Foundational Knowledge and Technologies for Extracellular RNA Research. Cell 177: 231-242.
54. Shi, Y., E. Mantuano, G. Inoue, W. M. Campana, and S. L. Gonias. 2009. Ligand binding to LRP1 transactivates Trk receptors by a Src family kinase-dependent pathway. Sci Signal 2: ra18.
55. Das, L., M. A. Banki, P. Azmoon, D. Pizzo, and S. L. Gonias. 2021. Enzymatically Inactive Tissue-Type Plasminogen Activator Reverses Disease Progression in the Dextran Sulfate Sodium Mouse Model of Inflammatory Bowel Disease. Am. J. Pathol. 191: 590-601.
56. Ogura, Y., D. K. Bonen, N. Inohara, D. L. Nicolae, F. F. Chen, R. Ramos, H. Britton, T. Moran, R. Karaliuskas, R. H. Duerr, J. P. Achkar, S. R. Brant, T. M. Bayless, B. S. Kirschner, S. B. Hanauer, G. Nuñez, and J. H. Cho. 2001. A frameshift mutation in NOD2 associated with susceptibility to Crohn's disease. Nature 411: 603-606.
57. Hugot, J. P., M. Chamaillard, H. Zouali, S. Lesage, J. P. Cézard, J. Belaiche, S. Almer, C. Tysk, C. A. O'Morain, M. Gassull, V. Binder, Y. Finkel, A. Cortot, R. Modigliani, P. Laurent-Puig, C. Gower-Rousseau, J. Macry, J. F. Colombel, M. Sahbatou, and G. Thomas. 2001. Association of NOD2 leucine-rich repeat variants with susceptibility to Crohn's disease. Nature 411: 599-603.
58. Lesept, F., A. Chevilley, J. Jezequel, L. Ladépêche, R. Macrez, M. Aimable, S. Lenoir, T. Bertrand, L. Rubrecht, P. Galea, L. Lebouvier, K. U. Petersen, Y. Hommet, E. Maubert, C. Ali, L. Groc, and D. Vivien. 2016. Tissue-type plasminogen activator controls neuronal death by raising surface dynamics of extrasynaptic NMDA receptors. Cell Death Dis. 7.
59. Mantuano, E., M. S. Lam, M. Shibayama, W. M. Campana, and S. L. Gonias. 2015. The NMDA receptor functions independently and as an LRP1 co-receptor to promote Schwann cell survival and migration. J. Cell Sci. 128: 3478-3488.
60. Lambrecht, B. N., M. Vanderkerken, and H. Hammad. 2018. The emerging role of ADAM metalloproteinases in immunity. Nat. Rev. Immunol. 18: 745-758.
61. Gonias, S. L., and W. M. Campana. 2014. LDL receptor-related protein-1: A regulator of inflammation in atherosclerosis, cancer, and injury to the nervous system. Am. J. Pathol. 184: 18-27.
62. Fernandez-Castaneda, A., S. Arandjelovic, T. L. Stiles, R. K. Schlobach, K. A. Mowen, S. L. Gonias, and A. Gaultier. 2013. Identification of the low density lipoprotein (LDL) receptor-related protein-1 interactome in central nervous system myelin suggests a role in the clearance of necrotic cell debris. J. Biol. Chem. 288: 4538-4548.
63. Johnstone, R. M. 2006. Exosomes biological significance: A concise review. Blood Cells, Mol. Dis. 36: 315-321.
64. Jan, A., S. Rahman, S. Khan, S. Tasduq, and I. Choi. 2019. Biology, Pathophysiological Role, and Clinical Implications of Exosomes: A Critical Appraisal. Cells 8: 99.
65. Xu, L., L.-F. Wu, and F.-Y. Deng. 2019. Exosome: An Emerging Source of Biomarkers for Human Diseases. Curr. Mol. Med. 19: 387-394.
66. Gaultier, A., X. Wu, N. Le Moan, S. Takimoto, G. Mukandala, K. Akassoglou, W. M. Campana, and S. L. Gonias. 2009. Low-density lipoprotein receptor-related protein 1 is an essential receptor for myelin phagocytosis. J. Cell Sci. 122: 1155-1162.
67. Ogden, C. A., A. DeCathelineau, P. R. Hoffmann, D. Bratton, B. Fadok, V. A. Ghebrehiwet, and P. M. Henson. 2001. C1q and mannose binding lectin engagement of cell surface calreticulin and CD91 initiates macropinocytosis and uptake of apoptotic cells. J. Exp. Med. 194: 781-795.
68. Vandivier, R. W., C. A. Ogden, V. A. Fadok, P. R. Hoffmann, K. K. Brown, M. Botto, M. J. Walport, J. H. Fisher, P. M. Henson, and K. E. Greene. 2002. Role of Surfactant Proteins A, D, and C1q in the Clearance of Apoptotic Cells In Vivo and In Vitro: Calreticulin and CD91 as a Common Collectin Receptor Complex. J. Immunol. 169: 3978-3986.

69. Head, B. P., H. H. Patel, Y. M. Tsutsumi, Y. Hu, T. Mejia, R. C. Mora, P. A. Insel, D. M. Roth, J. C. Drummond, and P. M. Patel. 2008. Caveolin-1 expression is essential for N-methyl-D-aspartate receptor-mediated Src and extracellular signal-regulated kinase 1/2 activation and protection of primary neurons from ischemic cell death. FASEB J. 22: 828-840.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. An anti-inflammatory agent comprising a cellular prion protein (PrP^{C}), or an analogue thereof, wherein said anti-inflammatory agent interacts with NMDA-R/LRP1 receptor complex to regulate innate immunity and provide anti-inflammatory effect.

2. The anti-inflammatory agent of claim 1, wherein said PrP^{c} is a soluble PrP^{c}.

3. The anti-inflammatory agent of claim 1, wherein said PrP^{c} is a membrane-anchored PrP^{c}.

4. The anti-inflammatory agent of claim 1, wherein said PrP^{c} is an extracellular vesicle (EV)-associated PrP^{C}.

5. A method of regulating an innate immunity in a disease comprising administering a cellular prion protein (PrP^{C}), or an analogue thereof, that interacts with NMDA-R/LRP1 receptor complex to provide anti-inflammatory effect.

6. The method of claim 5, wherein said PrP^{c} is a soluble PrP^{c}.

7. The method of claim 5, wherein said PrP^{c} is a membrane-anchored PrP^{c}.

8. The method of claim 5, wherein said PrP^{c} is an extracellular vesicle (EV)-associated PrP^{C}.

9. The method of claim 5, wherein said disease is selected from the group consisting of inflammatory Bowel Disease, Rheumatoid Arthritis, Psoriasis, Chronic Pain Disorders, Neurodegenerative Disease, and Multiple Sclerosis.

10. A composition for regulating an innate immunity in a disease comprising the anti-inflammatory agent of claim 1.

11. The composition of claim 10, wherein said composition is used for treating a disease in which innate immunity plays an important role.

12. The composition of claim 11, wherein the disease is selected from the group consisting of inflammatory Bowel Disease, Rheumatoid Arthritis, Psoriasis, Chronic Pain Disorders, Neurodegenerative Disease, and Multiple Sclerosis.

13. A kit comprising
a. at least one anti-inflammatory agent of claim 1; and
b. instructions for treating or preventing inflammation in a patient in need thereof.

14. The kit of claim 13, wherein the anti-inflammatory agent is formulated in a pharmaceutically acceptable carrier or excipients for a proper administration, alone or in combination with one or more agent.

15. A kit comprising
a. at least one composition of claim 10; and
b. instructions for treating or preventing inflammation in a patient in need thereof.

16. The kit of claim 15, wherein the composition is formulated in a pharmaceutically acceptable carrier or excipients for a proper administration, alone or in combination with one or more agent.

17. A composition for regulating an innate immunity in a disease comprising the anti-inflammatory agent of claim 4.

18. The composition of claim 17, wherein said composition is used for treating a disease in which innate immunity plays an important role.

19. The composition of claim 18, wherein the disease is selected from the group consisting of inflammatory Bowel Disease, Rheumatoid Arthritis, Psoriasis, Chronic Pain Disorders, Neurodegenerative Disease, and Multiple Sclerosis.

20. A kit comprising
a. at least one composition of claim 17; and
b. instructions for treating or preventing inflammation in a patient in need thereof.
